(19) United States
(12) Patent Application Publication
(10) Pub. No.: US 2024/0165365 A1
(43) Pub. Date: May 23, 2024

(54) MEDICAL GAS SUPPLY DEVICE

(71) Applicant: TAIYO NIPPON SANSO CORPORATION, Tokyo (JP)

(72) Inventors: Hiroki Hashi, Tokyo (JP); Nanami MIKI, Tokyo (JP)

(73) Assignee: TAIYO NIPPON SANSO CORPORATION, Tokyo (JP)

(21) Appl. No.: 18/506,369

(22) Filed: Nov. 10, 2023

(30) Foreign Application Priority Data
Nov. 22, 2022 (JP) 2022-186109

(51) Int. Cl. A61M 16/12; A61M 16/00; A61M 16/20

(52) U.S. Cl. CPC A61M 16/122; A61M 16/022; A61M 16/208; A61M 2016/0033; A61M 2202/02; A61M 2202/0225

(57) ABSTRACT

The invention relates to a medical gas supply device that supplies hydrogen gas to inhalation gas, including: an inhalation gas path that is connected to an inhalation line on an artificial respirator side; a hydrogen gas introduction path that is connected to a hydrogen gas supplier; a merging point where these two paths merge; a tank that is provided downstream of the merging point, includes a gas inlet and a gas outlet, has a diameter larger than that of the inhalation gas path, and mixes inhalation gas and hydrogen gas to form a mixed gas; a mixed gas supply path that connects the gas outlet and a patient-side inhalation line; a flow meter that is provided in the inhalation gas path; a flow rate controller that is provided in the medical gas introduction path; and a control unit that is connected to the flow meter and the flow rate controller.

### BACKGROUND OF THE INVENTION

### 1. Field of the Invention

The present invention relates to a medical gas supply device, specifically, a medical gas supply device that supplies and mixes medical gas with the inhalation gas from an artificial respirator.

### 2. Description of the Related Art

There is conventionally a hydrogen-mixed gas where hydrogen as a medical gas is merged in a tank and mixed with the inhalation gas from an artificial respirator, and this hydrogen-mixed gas is supplied to the patient (see, for example, Japanese Patent No. 5631524).

### SUMMARY OF THE INVENTION

Typically, as to the flow rate of inhalation gas from the artificial respirator, the ventilation mode thereof is determined according to the patient's respiratory condition, and for example, as shown in FIG. 5, the flow rate of inhalation gas repeats fluctuations in a set cycle. Therefore, when attempting to supply hydrogen-mixed gas to the patient, merely merging and mixing inhalation gas and hydrogen at the merging point could result in inconsistent concentrations of the medical gas in the mixed gas, necessitating high-precision control.

Especially when using hydrogen as a medical gas, there is a risk of explosive gas generation when mixing inhalation gas containing oxygen with hydrogen, and it is necessary to stabilize the hydrogen concentration at a concentration not exceeding 4 vol % of the explosive lower limit.

Therefore, the present invention aims to provide a medical gas supply device that can supply appropriate medical gas even when supplying medical gas at a constant flow rate without high-precision control to inhalation gas from an artificial respirator with fluctuating flow rates, and can also stabilize the concentration of medical gas.

To achieve the above object, a medical gas supply device of the present invention is a medical gas supply device that supplies medical gas to inhalation gas from an artificial respirator, including: an inhalation gas path that is connected to an inhalation line on an artificial respirator side; a medical gas introduction path that is connected to a medical gas supplier; a merging point where the inhalation gas path and the medical gas introduction path merge; a tank-shaped structure that is provided downstream of the merging point, includes a gas inlet and a gas outlet, has a diameter larger than a flow path diameter of the inhalation gas path, and mixes inhalation gas and medical gas to form a mixed gas; a mixed gas supply path that connects the gas outlet and a patient-side inhalation line; a flow meter that is provided in the inhalation gas path; a flow rate controller that is provided in the medical gas introduction path; and a control unit that is connected to the flow meter and the flow rate controller, wherein the control unit controls the flow rate controller so that the mixed gas has a predetermined concentration based on the flow rate over a predetermined time measured by the flow meter.

In addition, the flow meter can preferably detect a direction of gas flow.

In addition, the inhalation gas path is preferably provided with a check valve. Moreover, preferably, a molecular weight of the medical gas is smaller than a molecular weight of the inhalation gas, the tank-shaped structure is provided with the gas inlet on an upper surface thereof and the gas outlet on a lower surface thereof, and the medical gas in such cases is hydrogen.

Moreover, preferably, a molecular weight of the medical gas is larger than a molecular weight of the inhalation gas, the tank-shaped structure is provided with the gas inlet on a lower surface thereof and the gas outlet on an upper surface thereof, and the medical gas in such cases is carbon dioxide.

Furthermore, the tank-shaped structure is preferably provided with a gas diffuser on the gas inlet side thereof.

Further, the gas diffuser is preferably a baffle plate or a punched metal sheet that faces the gas inlet of the tank-shaped structure with a gap in between, and furthermore, the gas diffuser preferably include a cylindrical portion that extends from the gas outlet to the gas inlet side and has a distal end surface facing the gas inlet with a gap in between, and an opening that is provided in a side surface of the cylindrical portion.

According to the medical gas supply device of the present invention, the inhalation gas path is provided with a flow meter, and the medical gas introduction path is provided with a flow rate controller, and a control unit is provided that controls the flow rate controller so that hydrogen gas concentration in the mixed gas has a predetermined concentration based on the flow rate of inhalation gas over a predetermined time measured by the flow meter. This allows for the supply of medical gas at an appropriate flow rate to the inhalation gas, which fluctuates repeatedly.

In detail, based on the flow rate of inhalation gas measured by the flow meter over a predetermined time, the control unit calculates the average value of the flow rate of inhalation gas over a fixed time and calculates the flow rate of medical gas to be supplied from this average value. The flow rate controller then controls the flow rate of medical gas, supplying the calculated flow rate of medical gas to the inhalation gas. This allows for the provision of appropriate medical gas to the inhalation gas even when supplying medical gas at a constant flow rate without the need for high-precision control.

Furthermore, at the merging point where the inhalation gas path and the medical gas introduction path merge, the mixed gas, which has an inconsistent concentration of medical gas, is introduced into a tank-shaped structure with a larger diameter than the flow path diameter of the inhalation gas path. As a result, this gas flow generated within this tank-shaped structure eliminates the inconsistency in concentration of the medical gas. This allows for the delivery of mixed gas capable of maximizing the efficacy of the medical gas to the patient, and the oxygen concentration stabilizes as well, so that there is no risk of the patient inhaling a mixed gas with a low oxygen concentration.

Additionally, if the flow meter can detect the direction of gas flow, or if the inhalation gas path is provided with a check valve, even if the inlet-side connection portion (connection portion to be connected to the inhalation line of the artificial respirator) and the outlet-side connection portion (connection portion to be connected to the patient's inhalation line) of the medical gas supply device are mistakenly connected in reverse, the flow meter can detect errors to output an alert, or the check valve can block the flow of mixed gas to the patient's inhalation line.

Moreover, when the molecular weight of the medical gas is smaller than the molecular weight of the inhalation gas, for example, in the case of hydrogen, if the tank-shaped structure is provided with a gas inlet on the upper surface thereof and a gas outlet on the lower surface thereof, the smaller molecular weight medical gas rises against the downward gas flow from the upper surface gas inlet to the lower surface gas outlet. As a result, an upward gas flow occurs, ensuring that the mixed gas is well stirred and the concentration of the medical gas remains stable. Especially when the medical gas is hydrogen, it is possible to prevent the hydrogen gas from accumulating at the top of the tank structure, eliminating the risk of explosive gas formation.

Moreover, when the molecular weight of the medical gas is larger than the molecular weight of the inhalation gas, for example, in the case of carbon dioxide, if the tank-shaped structure is provided with a gas inlet on the lower surface thereof and a gas outlet on the upper surface thereof, the larger molecular weight medical gas descends against the upward gas flow from the lower surface gas inlet to the upper surface gas outlet. As a result, a downward gas flow occurs, ensuring that the mixed gas is well stirred and the concentration of medical gas remains stable.

Furthermore, by providing the tank-shaped structure with a gas diffuser on the gas inlet side thereof, the mixed gas introduced into the tank-shaped structure is well diffused, ensuring that the concentration of medical gas remains stable.

### BRIEF DESCRIPTION OF THE DRAWINGS

FIG. 1 is a schematic diagram showing the structure of a medical gas supply device according to the first embodiment of the present invention;

FIG. 2 is a cross-sectional perspective view showing a tank-shaped structure according to the first embodiment of the present invention:

FIG. 3 is a cross-sectional view of the same tank-shaped structure;

FIG. 4 is a cross-sectional view along line IV-IV of FIG. 3;

FIG. 5 is a graph measuring the flow rate of inhalation gas from an artificial respirator:

FIG. 6 is a graph measuring the hydrogen flow rate and concentration:

FIG. 7 is a schematic diagram showing the structure of a medical gas supply device according to the second embodiment of the present invention;

FIG. 8 is a cross-sectional perspective view showing a tank-shaped structure according to the second embodiment of the present invention:

FIG. 9 is a cross-sectional view of the same tank-shaped structure:

FIG. 10 is a cross-sectional view along line X-X of FIG. 9;

FIG. 11 is a cross-sectional perspective view showing a third embodiment of the tank-shaped structure; and

FIG. 12 is a cross-sectional view of the same tank-shaped structure.

### DESCRIPTION OF THE PREFERRED EMBODIMENTS

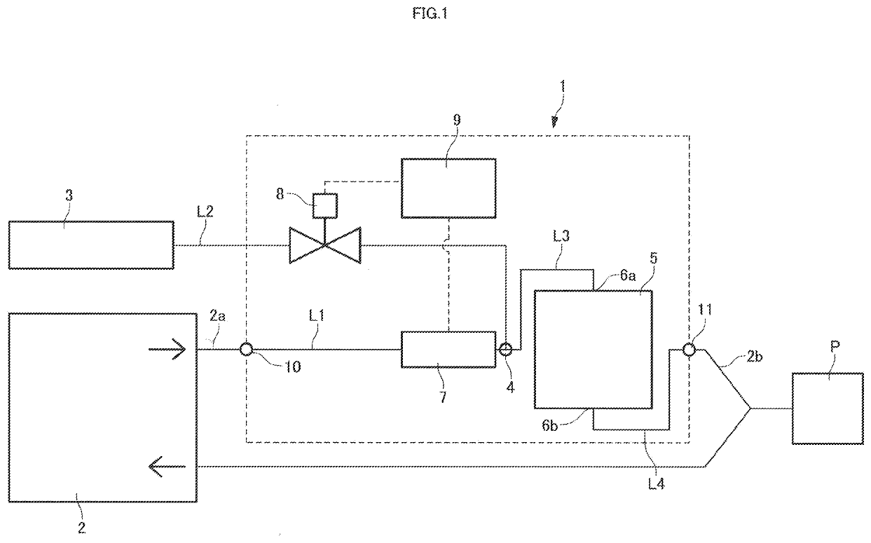

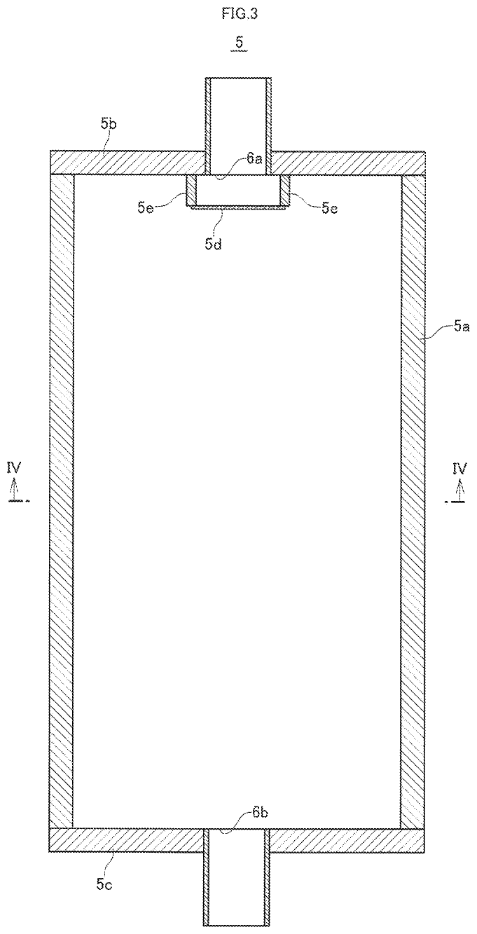

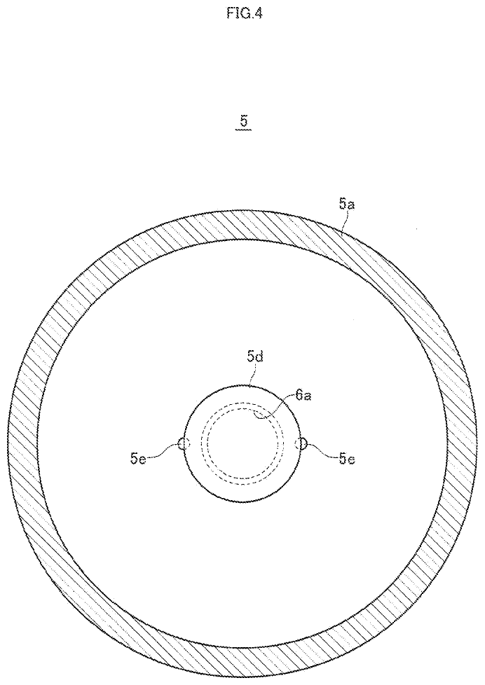

FIGS. 1 to 6 each show a first embodiment of the medical gas supply device according to the present invention. The medical gas supply device 1 is a device that supplies and mixes hydrogen gas (medical gas of the present invention) with inhalation gas from an artificial respirator 2, for example, air, oxygen, or a mixed gas of air and oxygen. The mixed gas derived from the medical gas supply device 1 is used for treating patient P.

This medical gas supply device 1, as shown in FIG. 1, includes an inhalation gas path L1 that is connected to the inhalation line 2a of the artificial respirator 2, a hydrogen gas introduction path L2 (medical gas introduction path of the present invention) that is connected to the hydrogen gas supplier 3 (medical gas supplier of the present invention), a merging point 4 where the inhalation gas path L1 and the hydrogen gas introduction path L2 merge, a tank 5 (tank-shaped structure of the present invention) that is provided downstream of the merging point 4, a mixed gas path L3 that connects the gas inlet 6a provided in the tank 5 and the merging point 4, and a mixed gas supply path L4 that connects the gas outlet 6b provided in the tank 5 and the patient-side inhalation line 2b. Additionally, the inhalation gas path L1 is provided with a flow meter 7 that measures the flow rate of inhalation gas, and the hydrogen gas introduction path L2 is provided with a flow rate controller 8 that controls the flow rate of hydrogen gas, with both the flow meter 7 and flow rate controller 8 connected to a control unit 9.

The control unit 9 controls the flow rate controller 8 so that the hydrogen gas concentration in the mixed gas has a predetermined concentration based on the flow rate of inhalation gas over a predetermined time measured by the flow meter 7. In detail, based on the flow rate of inhalation gas measured by the flow meter 7 over a predetermined time, the control unit 9 calculates the average value of the flow rate of inhalation gas over a fixed time and calculates the flow rate of hydrogen gas to be supplied from this average value. The flow rate controller 8 then controls the flow rate of hydrogen gas, supplying the calculated flow rate of hydrogen gas to the inhalation gas. This allows for the supply of hydrogen gas to the inhalation gas at the appropriate flow rate.

Furthermore, since the flow meter 7 can detect the direction of gas flow, it outputs an alert if a reverse gas flow, or negative flow rate, occurs.

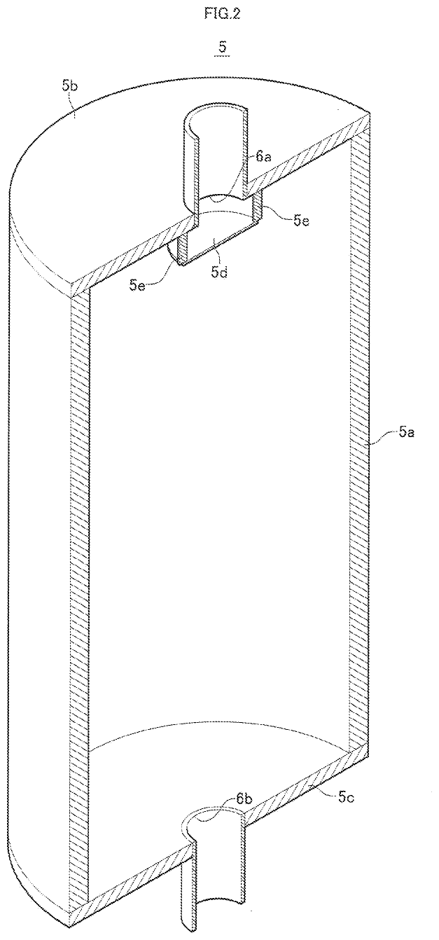

The tank 5, as shown in FIGS. 2 to 4, is formed by a vertically long cylindrical casing 5a with a larger diameter than the flow path diameter of the inhalation gas path L1, which is closed at the upper surface and lower surface thereof by a ceiling plate 5b and a bottom plate 5c, respectively. The central portion of the ceiling plate 5b is provided with the gas inlet 6a, and the central portion of the bottom plate 5c is provided with the gas outlet 6b.

Furthermore, inside the tank on the ceiling plate 5b, there is provided a baffle plate 5d (gas diffuser) that is arranged facing the gas inlet 6a with a gap in between. The baffle plate 5d is formed in a circular shape with a larger diameter than the gas inlet 6a, and the outer periphery of the baffle plate 5d and the tank inner side surface of the ceiling plate 5b are connected by two support members 5e, 5e.

When using the medical gas supply device 1 formed as described above, first, one connects the inhalation line 2a of the artificial respirator 2 and the inhalation gas path L1 via the first connection portion 10, and connects the inhalation line 2b on the patient P side and the mixed gas supply path L3 via the second connection portion 11.

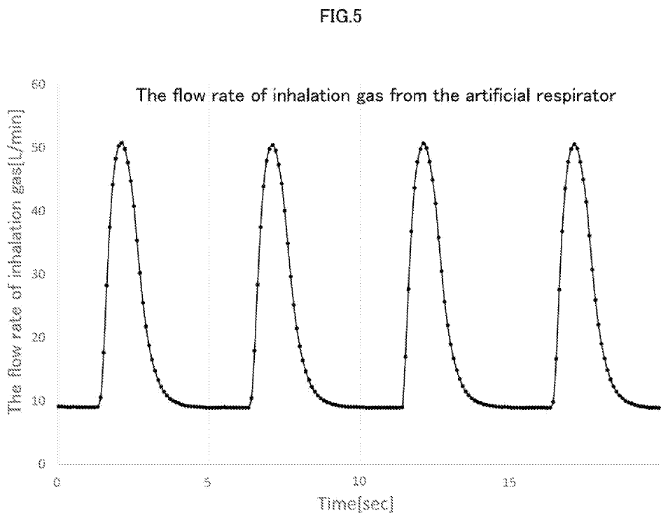

When artificial respiration is initiated for patient P, inhalation gas is introduced from the artificial respirator 2 into the inhalation gas path L1, and hydrogen gas is introduced from the hydrogen gas supplier 3 into the hydrogen gas introduction path L2. The inhalation gas introduced into the inhalation gas path L3, as shown in FIG. 5, has a ventilation mode determined according to the respiratory state of patient P, and the flow rate of inhalation gas is introduced into the merging point 4 in a state of fluctuating at a set cycle.

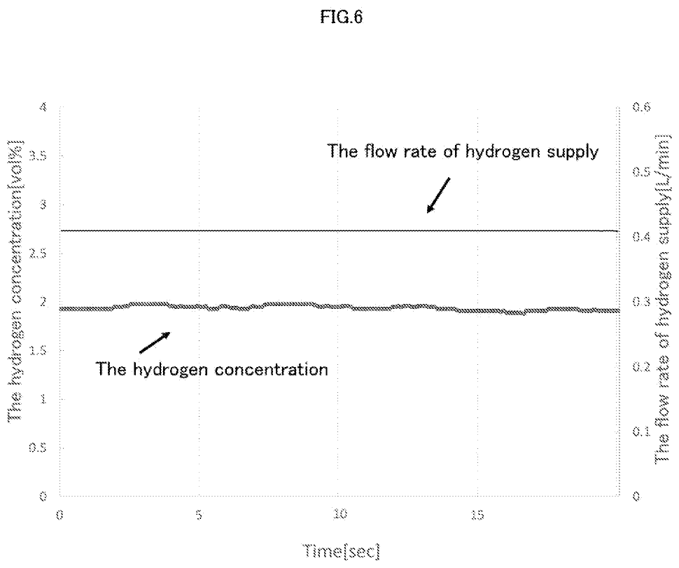

On the other hand, the hydrogen gas introduced into the hydrogen gas introduction path L2 is calculated by the control unit 9 to have an optimal flow rate corresponding to the flow rate of inhalation gas, and is introduced into the merging point 4 at this optimal flow rate by the flow rate controller 7. For example, for the flow rate of inhalation gas as shown in FIG. 5, the control unit 9 calculates that the average flow rate of the inhalation gas is 20.5 L/min. If, for such flow rate of inhalation gas, one tries to obtain a mixed gas with a hydrogen concentration of 2 vol % as shown in the hydrogen supply-concentration graph of FIG. 6, it is judged that 20 L/min×0.02=410 mL/min of hydrogen gas should be supplied, and the hydrogen gas flow rate is controlled to the optimal flow rate by the flow rate controller 7.

Merging at the merging point 4, the mixed gas of the inhalation gas introduced into the mixed gas path L3 and hydrogen gas is introduced into the tank 5 from the gas inlet 6a in a state of inconsistency in the hydrogen gas concentration.

The mixed gas introduced into the tank 5 from the gas inlet 6a comes into contact with the baffle plate 5d, passes through the gap provided between the baffle plate 5d and the gas inlet 6a, and flows downward while diffusing in the peripheral wall direction of the casing 5a. Against this downward gas flow, hydrogen gas with a smaller molecular weight than the inhalation gas rises, causing an upward gas flow, thus the mixed gas is well stirred and the hydrogen concentration can be stabilized evenly. Furthermore, it is possible to prevent the hydrogen gas from accumulating at the top of the tank 5, eliminating the risk of explosive gas formation.

According to the present medical gas supply device 1, even if hydrogen gas is supplied at a constant flow rate without high-precision control to inhalation gas from an artificial respirator 2 whose flow rate fluctuates, it is possible to provide hydrogen gas at an appropriate flow rate and stabilize the hydrogen gas concentration. This allows for the delivery of mixed gas capable of maximizing the efficacy of the hydrogen gas to the patient, and the oxygen concentration stabilizes as well, so that there is no risk of the patient inhaling a mixed gas with a low oxygen concentration.

Moreover, in the event that the medical gas supply device 1 is inadvertently connected such that the inhalation line 2b on the patient P side is connected to the first connection portion 10, and the inhalation line 2a of the artificial respirator 2 is connected to the second connection portion 11, a reverse gas flow results in a negative flow rate. Consequently, the flow meter 7 outputs an alert, preventing the malfunction of the medical gas supply device 1.

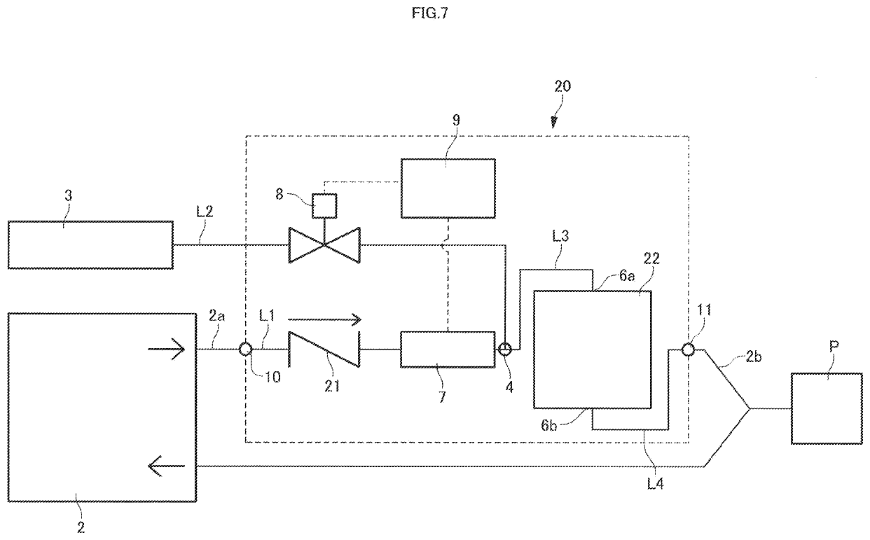

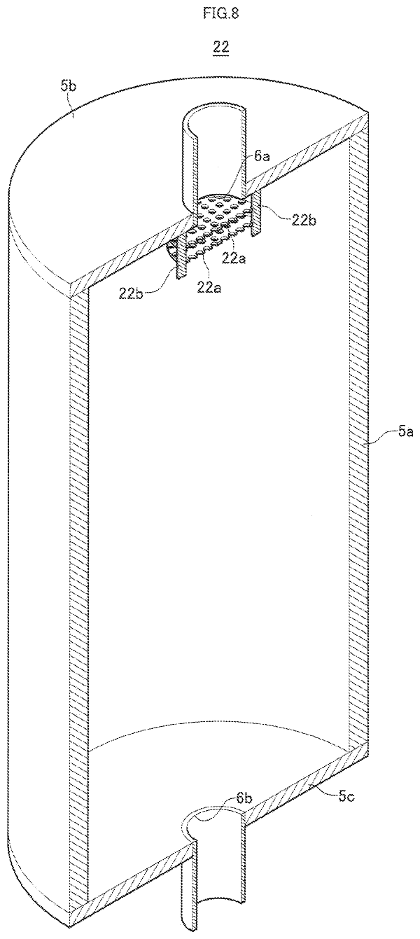

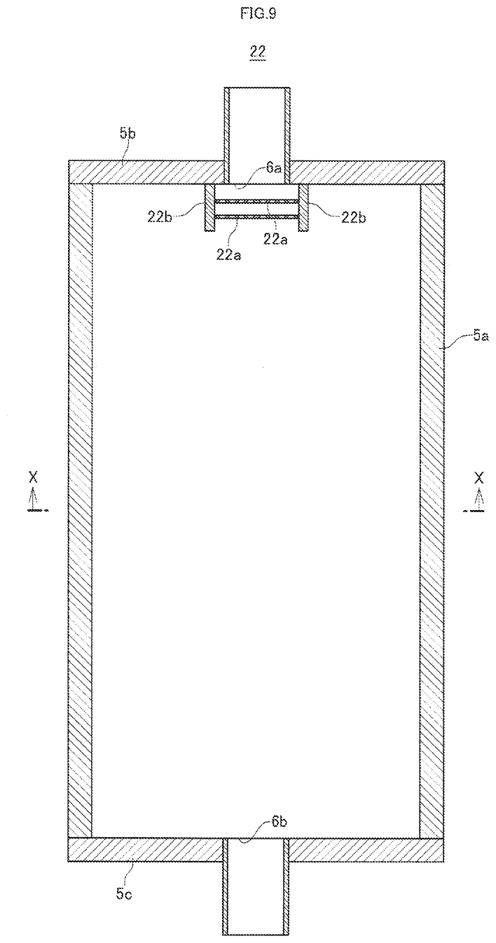

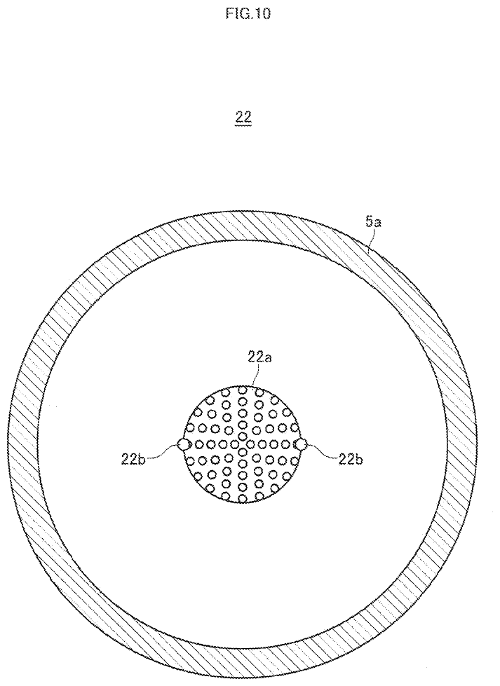

FIGS. 7 to 10 each show a second embodiment of the present invention. For components that are analogous to those of the first embodiment, the same reference numerals are attached, and detailed explanations thereof are omitted.

In the medical gas supply device 20 of the present embodiment, the inhalation gas path L1 is provided with a check valve 21. As a result, in the event that the medical gas supply device 20 is inadvertently connected such that the inhalation line 2b on the patient P side is connected to the first connection portion 10, and the inhalation line 2a of the artificial respirator 2 is connected to the second connection portion 11, the gas flow is interrupted by the check valve 21, prompting the artificial respirator 2 to issue an alert.

Furthermore, the tank 22 of the present embodiment is provided with two punched metal sheets 22a, 22a (gas diffusers) positioned inside the tank on the ceiling plate 5b, facing the gas inlet 6a with a gap in between. The punched metal sheets 22a, 22a are formed in a circular shape with a diameter larger than the gas inlet 6a, and are placed vertically with a gap in between. The outer periphery of each punched metal sheet 22a, 22a and the tank inner side surface of the ceiling plate 5b are connected by two support members 22b, 22b.

As such, the mixed gas introduced into the tank 22 from the gas inlet 6a comes into contact with the punched metal sheets 22a, 22a, passes through the gap provided between the upper punched metal sheet 22a and the gas inlet 6a and the gap between the two punched metal sheets 22a, 22a, and either flows downward while diffusing in the peripheral wall direction of the casing 5a, or flows downward through the multiple holes formed in the punched metal sheets 22a, 22a. Also, against this downward gas flow, hydrogen gas with a smaller molecular weight rises, causing an upward gas flow, thus the mixed gas is well stirred and the hydrogen concentration can be stabilized evenly. Furthermore, it is possible to prevent the hydrogen gas from accumulating at the top of the tank 22.

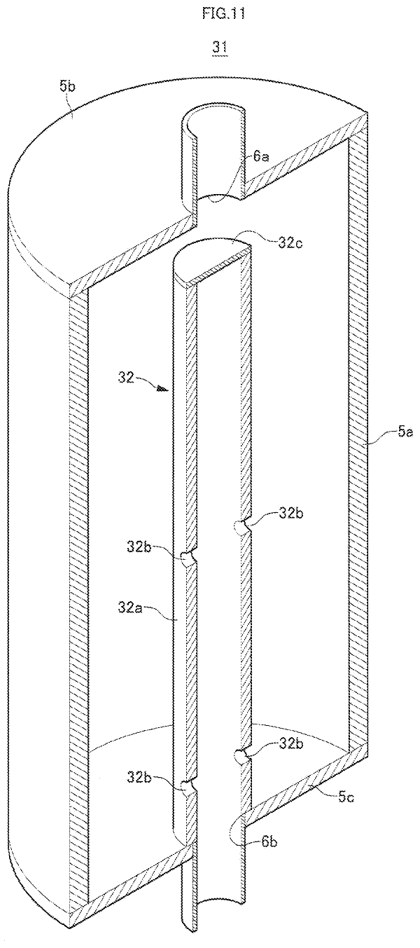

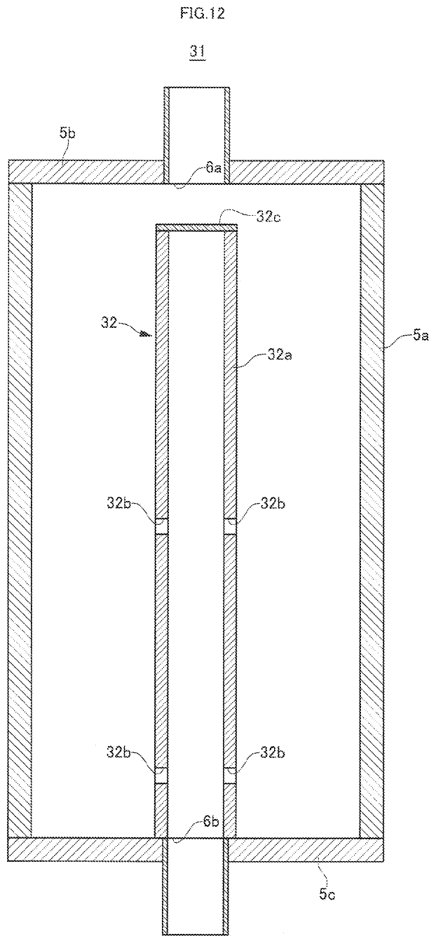

FIGS. 11 and 12 each show a third embodiment of the tank. For components that are analogous to those of the first embodiment, the same reference numerals are attached, and detailed explanations thereof are omitted.

The tank 31 of the present embodiment includes a gas diffuser 32 that has a cylindrical portion 32a extending from the gas outlet 6b to the gas inlet 6a side, and multiple openings 32b provided in the side surface of the cylindrical portion 32a. The inner diameter of the cylindrical portion 32a is formed approximately the same as that of the mixed gas supply path L4 connected to the gas outlet 6b, and is attached to the bottom plate 5c so as to communicate with the mixed gas supply path L4. The distal end surface 32c thereof faces the gas inlet 6a with a gap in between.

As a result, the mixed gas introduced into the tank 31 from the gas inlet 6a comes into contact with the distal end surface 32c of the cylindrical portion 32a, passes through the gap provided between the distal end surface 32c and the gas inlet 6a, and flows downward while diffusing in the peripheral wall direction of the casing 5a. Also, against this downward gas flow, hydrogen gas with a smaller molecular weight rises, causing an upward gas flow, thus the mixed gas is well stirred and the hydrogen concentration can be stabilized evenly. Furthermore, the mixed gas is introduced through the multiple openings 32b into the inside of the cylindrical portion 32a and is introduced into the mixed gas supply path L4.

In the aforementioned embodiments, hydrogen gas, which has a smaller molecular weight than inhalation gas, is used as the medical gas. However, the medical gas supply device of the present invention is not limited to this. It can also be applied to those using carbon dioxide, for example, which has a larger molecular weight than the inhalation gas, as the medical gas.

In this case, the lower surface of the tank is provided with a gas inlet, and the upper surface with a gas outlet. The mixed gas containing carbon dioxide introduced into the tank from the gas inlet flows upward to the gas outlet provided on the upper surface of the tank. Carbon dioxide, which has a larger molecular weight than the inhalation gas, descends against the upward gas flow. As a result, a downward gas flow occurs, ensuring that the mixed gas is well stirred and the concentration of carbon dioxide remains consistently stable.

Thus, the medical gas supply device of the present invention can be applied to any medical gas by changing the positions of the gas inlet and gas outlet provided in the tank according to the molecular weight of the medical gas used.

Even in cases where no check valve is provided in the inhalation gas path L1, as in the first embodiment, a tank 5 with the structure shown in the second embodiment and third embodiments may be used. Similarly, even in cases where a check valve is provided in the inhalation gas path L1, as in the second embodiment, a tank 5 with the structure shown in the first embodiment and third embodiments may be used. Furthermore, the shape of the gas diffuser provided in the tank is not limited to the aforementioned embodiments and can be optional. Moreover, there is no problem even if no gas diffuser is provided in the tank.

1. A medical gas supply device that supplies medical gas to inhalation gas from an artificial respirator, including:
an inhalation gas path that is connected to an inhalation line on an artificial respirator side;
a medical gas introduction path that is connected to a medical gas supplier;
a merging point where the inhalation gas path and the medical gas introduction path merge;
a tank-shaped structure that is provided downstream of the merging point, includes a gas inlet and a gas outlet, has a diameter larger than a flow path diameter of the inhalation gas path, and mixes inhalation gas and medical gas to form a mixed gas;
a mixed gas supply path that connects the gas outlet and a patient-side inhalation line;
a flow meter that is provided in the inhalation gas path;
a flow rate controller that is provided in the medical gas introduction path; and
a control unit that is connected to the flow meter and the flow rate controller, wherein
the control unit controls the flow rate controller so that the mixed gas has a predetermined concentration based on the flow rate over a predetermined time measured by the flow meter.

2. The medical gas supply device according to claim 1, wherein the flow meter can also detect a detect a direction of gas flow.

3. The medical gas supply device according to claim 1, wherein the inhalation gas path is provided with a check valve.

4. The medical gas supply device according to claim 1, wherein
a molecular weight of the medical gas is smaller than a molecular weight of the inhalation gas, and
the tank-shaped structure is provided with the gas inlet on an upper surface thereof and the gas outlet on a lower surface thereof.

5. The medical gas supply device according to claim 4, wherein the medical gas is hydrogen.

6. The medical gas supply device according to claim 1, wherein
a molecular weight of the medical gas is larger than a molecular weight of the inhalation gas, and
the tank-shaped structure is provided with the gas inlet on a lower surface thereof and the gas outlet on an upper surface thereof.

7. The medical gas supply device according to claim 6, wherein the medical gas is carbon dioxide.

8. The medical gas supply device according to claim 1, wherein the tank-shaped structure is provided with a gas diffuser on the gas inlet side thereof.

9. The medical gas supply device according to claim 8, wherein the gas diffuser is a baffle plate that faces the gas inlet of the tank-shaped structure with a gap in between.

10. The medical gas supply device according to claim 8, wherein the gas diffuser is a punched metal plate that faces the gas inlet of the tank-shaped structure with a gap in between.

11. The medical gas supply device according to claim 8, wherein the gas diffuser include a cylindrical portion that extends from the gas outlet to the gas inlet side and has a distal end surface facing the gas inlet with a gap in between, and an opening that is provided in a side surface of the cylindrical portion.

12. The medical gas supply device according to claim 2, wherein
a molecular weight of the medical gas is smaller than a molecular weight of the inhalation gas, and
the tank-shaped structure is provided with the gas inlet on an upper surface thereof and the gas outlet on a lower surface thereof.

13. The medical gas supply device according to claim 3, wherein
a molecular weight of the medical gas is smaller than a molecular weight of the inhalation gas, and
the tank-shaped structure is provided with the gas inlet on an upper surface thereof and the gas outlet on a lower surface thereof.

14. The medical gas supply device according to claim 12, wherein the medical gas is hydrogen.

15. The medical gas supply device according to claim 13, wherein the medical gas is hydrogen.

16. The medical gas supply device according to claim 2, wherein
a molecular weight of the medical gas is larger than a molecular weight of the inhalation gas, and
the tank-shaped structure is provided with the gas inlet on a lower surface thereof and the gas outlet on an upper surface thereof.

17. The medical gas supply device according to claim 3, wherein
a molecular weight of the medical gas is larger than a molecular weight of the inhalation gas, and
the tank-shaped structure is provided with the gas inlet on a lower surface thereof and the gas outlet on an upper surface thereof.

18. The medical gas supply device according to claim 16, wherein the medical gas is carbon dioxide.

19. The medical gas supply device according to claim 17, wherein the medical gas is carbon dioxide.
